(19) United States
(12) Patent Application Publication
(10) Pub. No.: US 2025/0228236 A1
(43) Pub. Date: Jul. 17, 2025

(54) EXTRACORPOREAL ORGAN SUPPORT SYSTEM

(71) Applicant: Miromatrix Medical Inc., Eden Prairie, MN (US)

(72) Inventors: Aleksandr Katane, Victoria, MN (US); Dmitrii Pokhil, Eden Prairie, MN (US); Shawn Riesgraf, Carver, MN (US); Aron Stumbras, Eden Prairie, MN (US)

(21) Appl. No.: 18/702,789

(22) PCT Filed: Oct. 18, 2022
(86) PCT No.: PCT/US2022/078272

§ 371 Date: Apr. 18, 2024

Related U.S. Application Data
(60) Provisional application No. 63/257,004, filed on Oct. 18, 2021.

(51) Int. Cl. A01N 1/143; A01N 1/12; A01N 1/162

(52) U.S. Cl. CPC A01N 1/143; A01N 1/12; A01N 1/162

(57) ABSTRACT

A system (200) for supporting a patient organ can include a primary circuit (212b), a secondary circuit (212a), and a controller (214). The primary circuit can include an inlet configured to connect to the patient (54), an outlet configured to connect to the patient, and a primary pump (206a). The secondary circuit can be connected to the primary blood circuit and can include an enclosure (202) configured to support an organ (50) therein in a blood flow, a secondary pump, a gas transfer unit (208), and a secondary sensor (238a). The controller can be configured to operate the gas transfer unit based on the secondary sensor signal. The organ may be a bio-engineered organ. In an example, the organ is a liver and operates to perform functions of a liver in place of or in support of the liver of the patient.

### CLAIM OF PRIORITY

This patent application claims the benefit of priority, under 35 U.S.C. Section 119(e), to Aleksandr Katane, U.S. Patent Application Ser. No. 63/257,004, entitled “EXTRACORPOREAL ORGAN SUPPORT SYSTEM,” filed on Oct. 18, 2021, which is hereby incorporated by reference herein in its entirety.

### BACKGROUND

Organ transplants are common across the United States and throughout the world. Organs such as livers, kidneys, pancreas, lungs and hearts are commonly transplanted to prolong the life of the recipients. However, there is often a shortage of organs, creating a demand and a waiting list for the organs. One solution to this shortage is to use biologically engineered organs and alternative tissues, where organs are stripped of their cells and the recipient's cells can be perfused into the biologically engineered organ, to help prevent rejection.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 illustrates an overview of devices and systems involved in stages of transplanting a biologically engineered organ.

FIG. 2 illustrates a schematic view of a system for growth and support of a bio-engineered organ.

FIG. 3 illustrates a schematic view of a system for growth and support of a bio-engineered organ.

FIG. 4 illustrates a schematic view of a method of operating a system for growth and support of a bio-engineered organ.

FIG. 5 illustrates a block diagram of architecture for an example computing system used.

FIG. 6 illustrates a schematic view of a system for growth and support of a bio-engineered organ.

### DETAILED DESCRIPTION

Biologically engineered organs (BEOs) and advanced bio-engineered tissues (ATs) can help reduce rejection, helping to reduce organ wait times. BEOs can also help to reduce wait times by making use of organs of different species. In some examples, a BEO or AT can be connected to a patient extracorporeally for testing of the AT or BEO before implantation or for providing additional functional support to the patient who may be experiencing organ failure or reduced organ function. In such an example, the organ can be connected to the patient using a support system for monitoring conditions of the patient and AT or BEO.

For example, while the AT or BEO is connected to the patient, the system can actively monitor the housed AT or BEO for function to ensure that it meets specifications prior to implantation while helping to improve the health of the patient, optionally prior to implantation. The system can also monitor health of the AT or BEO and health of the patient and patient organ while the AT or BEO is connected to the patient to help ensure organ compatibility. Also, these functions can be performed by the system automatically. By automatically adjusting operation of the system or automatically monitoring health of the patient or organ(s), the system can help to reduce labor requirements for supporting a patient using a BEO or AT.

The above discussion is intended to provide an overview of subject matter of the present patent application. It is not intended to provide an exclusive or exhaustive explanation of the invention. The description below is included to provide further information about the present patent application.

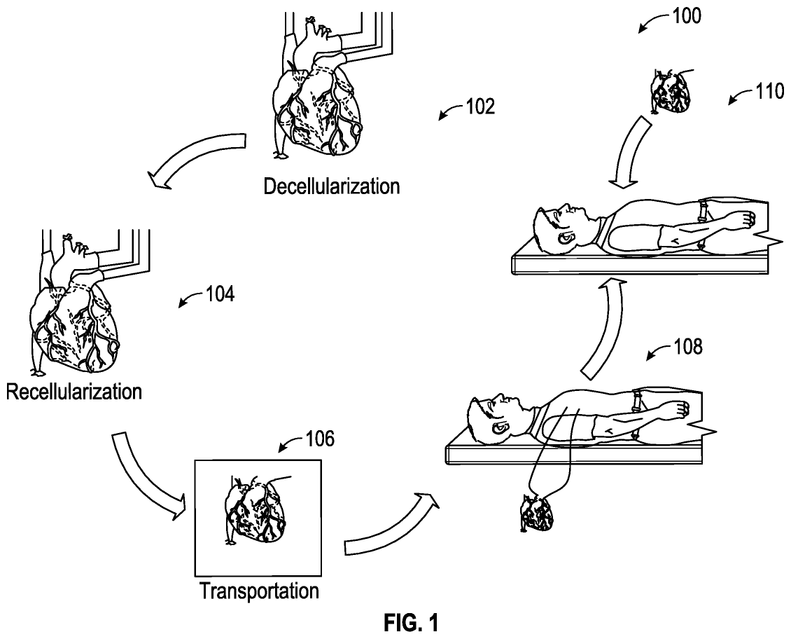

FIG. 1 illustrates an overview of devices and systems involved in stages of transplanting a BEO in accordance with at least one example of the present disclosure.

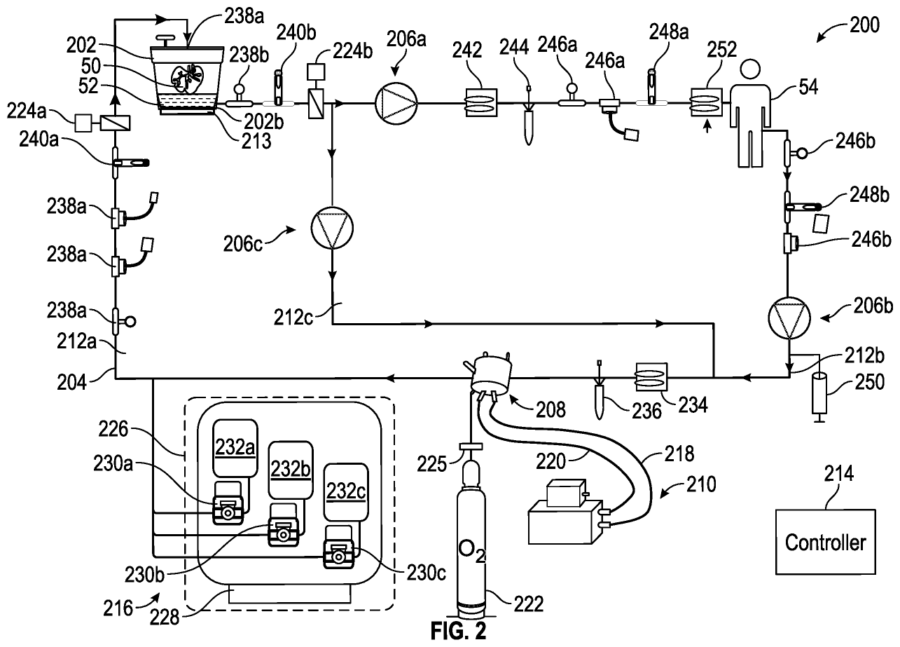

An example procedure 100 shows various stages that can be included in a transplantation procedure. At a stage 102, an organ can be received from a donor human or other species, such as pig, sheep, bovine, or the like. The organ can be decellularized in a laboratory under carefully controlled conditions, where all (or substantially all) cellular material can be removed from the organ. Following decellularization, the organ can be recellularized at a stage 104, whereby cells from the donor (or other sources) can be added to the organ and the organ can be functionally tested and otherwise prepared for implantation. Once it is determined that the BEO is ready for transplant, the BEO can be prepared for transportation at stage 106. At step 108, the BEO or AT can be connected to the patient, such as to provide patient support or to test organ compatibility. Then, at step 110, the BEO can be implanted into the recipient in a transplant operation. Further details of several of these steps are discussed in further detail below with respect to FIGS. 2-6.

FIG. 2 illustrates a schematic view of a system 200 for support and maintenance of a patient using a bio-engineered organ (BEO). The system 200 can include an enclosure 202, a blood circuit 204, a pump 206 (including a pump 206a, a pump 206b, and a pump 206c), a gas transfer system 208, a heating/cooling system 210. The circuit 204 can include a secondary circuit 212a, a primary circuit 212b, and a bypass 212c. The system 200 can also include a controller 214 and an injection system 216. Also shown in FIG. 2 is an organ 50, which can be a liver, for example, blood 52, which can be a fluid, and a patient 54.

The enclosure 202 can be an enclosure configured to support the organ 50 therein in blood or media flow. The enclosure 202 can include a blood inlet 202a and a blood outlet 202b to receive the blood flow through the enclosure and through the organ 50. The enclosure 202 can be a rigid or semi-rigid container, enclosure, or housing made of materials such as one or more of metals, plastics, foams, elastomers, ceramics, composites, combinations thereof, or the like, such as polycarbonate. The enclosure 202 can be transparent or translucent to provide visibility of the organ 50 therein for operators or technicians. Optionally, the enclosure 202 can be configured to limit exposure of the organ 50 to light. The enclosure 202 can include one or more latches, fasteners, or the like to resealably close a lid. In some examples, the enclosure 202 can include one or more hinges. The enclosure 202 can include one or more insulative layers to help thermally isolate contents of the enclosure 202 from ambient conditions. The enclosure 202 can include a seal between the lid and the container of the enclosure 202.

The enclosure 202 can be configured to receive and support a biologically engineered organ therein in a blood flow and/or bath. That is, the enclosure 202 can be shaped to support an organ therein. In some examples, the enclosure 202 can be organ-specific (such as kidney specific) such that the enclosure 202 is shaped to support the kidney (or other organ in other examples). In some examples, the organ-specific enclosure 202 can be removable and replaceable as required for an organ to be transported. The blood inlet 202a and the blood outlet 202b can include sterile-quick connects for disconnecting the enclosure 202 from the blood circuit 204.

The system 200 can also include a weight sensor 213 connected to the enclosure 202. The weight sensor 213 can be in communication with the controller 214 such as to transmit a weight signal thereto. The controller 214 can determine a weight or volume of fluid within the enclosure 202 and can optionally control various components of the system based on the weight or volume, such as the pumps 206.

The secondary circuit 212a of the blood circuit 204 can be connected to the blood inlet 202a and the blood outlet 202b and can be configured to transmit blood through the system 200 and its components. The bypass 212c can connect the secondary circuit 212a to the primary circuit 212b, such as to allow flow rates through the secondary circuit 212a and the primary circuit 212b to vary or be different.

The circuit 204 can be made of one or more types of tubing including rigid, semi-rigid, and flexible tubing on various materials (e.g., copper, steel, aluminum, plastics, silicone, or the like). Optionally, the tubing of the circuit 204 can be low-shed tubing or pharmacy-grade tubing. Additionally, the blood circuit 204 can be configured to have relatively few restriction points throughout the blood circuit 204 to help reduce pressure drop through the blood circuit 204, which can help increase an accuracy between pressure measured outside the organ 50 and at a pressure sensor and pressure at or within the organ 50.

The tubing or circuit 204 of the secondary circuit 212a can optionally connect directly to perfusion vessels of the organ 50, which can allow for independent vessel interfacing. Though blood is discussed generally with respect to FIG. 2, the blood circuit can be configured to handle one or more other fluids, such as media or perfusate. In some examples, patient blood can be used in the circuit 204.

The pumps 206a, 206b, and 206c can each be connected to the circuit 204 and can be configured to circulate blood through the blood circuit 204. The pumps 206 can be a positive displacement pump, a centrifugal pump, or an axial pump. The pumps 206 can be a low-shear pump to help limit damage to the fluid and can optionally be reversible. In some examples, the pump 206 can be a continuous type pump or a peristaltic type pump (with or without damping).

The pump 206a can be connected to the primary circuit 212b, such as downstream of the enclosure 202 and upstream of the patient 54. The pump 206b can be connected to the primary circuit 212b downstream of the patient 54. The pump 206c can be connected to the bypass 212c. The pumps 206a-206c can be connected to other portions of the blood circuit 204 in other examples. Optionally, the system can include more pumps or fewer pumps.

The gas transfer unit 208 can be connected to the secondary circuit 212a of the blood circuit 204 and can be configured to transfer gas to and from the blood, such as oxygen, nitrogen, argon, and carbon dioxide (CO2). The gas transfer unit 208 can be either an active gas transfer unit or a passive gas transfer unit configured to maintain, for example, oxygen and CO2 at a desired concentration range within the blood. In some examples, the gas transfer unit 208 can manage other gasses conducive to organ support. The gas transfer unit 208 can be located upstream of the enclosure 202 such as to exchange gasses of the blood before it enters the organ 50. The gas transfer unit 208 can optionally include a heat exchanger configured to exchange heat between blood of the blood circuit 204 and the heating and cooling system 210 such as to maintain the temperature of the blood before it enters the organ 50. Optionally, a separate heat exchanger can be used in the system 200. Optionally, the gas transfer unit 208 can be an oxygenator.

The heating/cooling system 210 can be connected to the blood circuit 204 to exchange heat with the blood such that the blood is delivered to the enclosure 202 within a target temperature range. The heating/cooling system 210 can be connected to the gas transfer unit 208 such as to allow the heat exchanger of the gas transfer unit 208 to exchange heat with the blood in the blood circuit 204, such as the secondary circuit 212a. For example, a supply line 218 and a return line 220 can connect the heating/cooling system 210 to the gas transfer unit 208, such as to the heat exchanger of the gas transfer unit 208.

The heating/cooling system 210 can be a heating system in some examples and can be a cooling system in some examples. In some examples, the system 210 can include discrete heating and cooling systems. For example, the system 210 can include a resistive heater, fan, and heat exchanger. In other examples, the system 210 can be, for example, a refrigerant heat pump including a compressor, a condenser, an evaporator, one or more expansion valves, and a reversing valve. In some examples the system 210 can be a thermoelectric heating and cooling device (Peltier device). In some examples, the system can include a refrigeration cooling system and a resistive electric heating device. In any of these examples, the system 210 can include a heat exchanger configured to exchange heat with the blood indirectly. Optionally, the system 210 can include an emergency cooling system. Optionally, the gas transfer unit 208 can include or can be connected to one or more gas tanks 222. The gas tank 222 can be configured to deliver one or more of Carbon Dioxide, Dioxygen, Nitrogen, or Argon to the gas transfer unit 208. The gas tank 222 can be connected to the gas transfer unit 208 by a filter 225.

The system 200 can optionally include or can be connected to a power source that can be configured to power the pumps 206, the heating/cooling system 210, the gas transfer unit 208, the various sensors of the system 200, the controller, a user interface 254, and/or other components of the system 200. Optionally, the power source can be an uninterruptable power supply to help provide constant operation of the system 200 even during loss of input power.

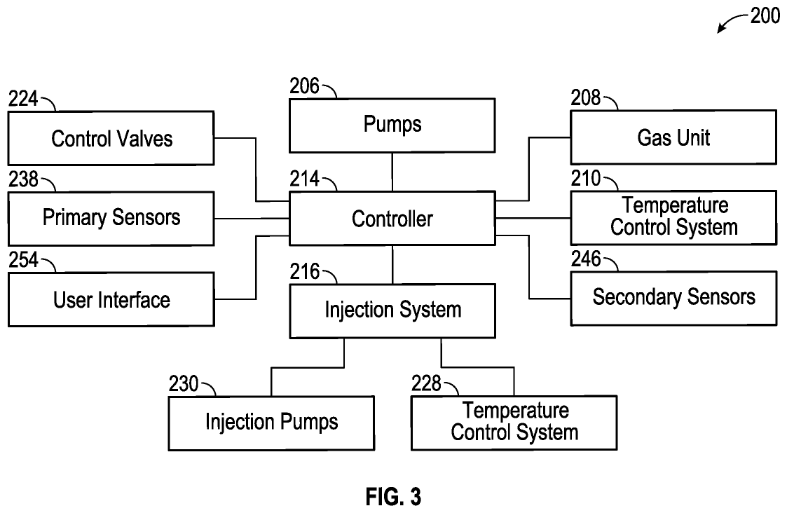

The controller 214 can be a programmable controller, such as a single or multi-board computer, a direct digital controller (DDC), a programmable logic controller (PLC), or the like. In other examples the controller 214 can be any computing device, such as a handheld computer, for example, a smart phone, a tablet, a laptop, a desktop computer, or any other computing device including a processor, memory, and communication capabilities. The controller 214 can generally be configured to control operations of the systems 200, such as by controlling operation of the pumps 206, the gas transfer unit 208, the heating/cooling system 210, the power source, any sensor (such as those discussed below), or the injection system 216. Various examples of how the controller 214 can control one or more components the system 200 to support or grow the organ 50 are discussed below with reference to FIG. 3.

The secondary circuit 212a can optionally include an inlet control valve 224a and an outlet control valve 224b. The inlet control valve 224a and the outlet control valve 224b can each be modulating valves or isolation valves in communication with the controller and can be operated thereby, such as to isolate the enclosure 202 and the organ 50, such as for removal of the enclosure 202 and the organ 50 from the system 200. Optionally the inlet control valve 224a and the outlet control valve 224b can be in communication with the controller 214 and the controller 214 can operate the inlet control valve 224a or the outlet control valve 224b to regulate flow through the enclosure 202 and the organ 50. The primary circuit 212b and the bypass 212c can also include one or more control valves.

The injection system 216 can include a housing 226, a heating/cooling system 228, pumps 230a-230c, and tanks 232a-232c. The injection system 216 can be configured to inject one or more nutrients or fluids into the blood circuit 204, such as the secondary circuit 212a. The discharge of each pump 230 can be connected or manifolded together and can be connected to the blood circuit 204, such as upstream of the blood inlet 202a. Optionally, the injection system 216 can connect to the blood circuit 204 in another location. Optionally, each pump 230 can include a check valve therein or associated therewith. Optionally, the pump can include an intrinsic check valve capability, such that the pumps cannot pump past each other when manifolded or connected together.

The enclosure 226 can include one or more walls and insulation, such as to provide a temperature-controlled environment within the enclosure 226. The enclosure 226 can made of materials such as one or more of metals, plastics, foams, elastomers, ceramics, composites, combinations thereof, or the like. The enclosure 226 can be transparent or translucent (or can include a transparent or translucent portion, e.g., window) to provide visibility of the pumps 230 and tanks 232. Optionally, the enclosure 226 can be configured to limit exposure of the tanks 232 to light. The enclosure 226 can include one or more latches, fasteners, or the like to resealably close a door or lid. In some examples, the enclosure 226 can include one or more hinges to connect a door. The enclosure 226 can include a seal between a door and the container of the enclosure 226.

The pumps 230 can each be configured to pump fluid therethrough and to discharge fluid to the blood circuit 204. Each of the pumps 230 can be any of the pump types discussed above with respect to the pumps 206. The tanks 232 can each be a storage container made of one or more of metals, plastics, foams, elastomers, ceramics, composites, combinations thereof, or the like. The tanks 232 can each be configured to store fluids to be injected into the blood circuit 204 as needed, or as determined by an operator or the controller 214. Though three tanks and pumps are shown the injection system 216 can include 1, 2, 4, 5, 6, 7, 8, 9, 10, or the like tanks or pumps.

The tanks 232 can be configured to store and the pumps 230 can be delivered to pump one or more of cell culture media, cells, glutamine, glucose, buffer, sodium chloride, essential amino acids, non-essential amino acids, drugs, ammonia (ammonium chloride), HEPES (C₈H₁₈N₂O₄S), Sodium Bicarbonate, Insulin, Epinephrine, Albumin, linoleic acid, dexamethasone, or glucagon. The drugs can be one or more of (R)-Warfarin, (S)-Mephenytoin, Acetaminophen, Aprepitant, Azole, Benzodiazepines, Beta, Caffeine, Calcium, Carbamazepine, Celecoxib, Clarithromycin, Codeine, Cyclosporine, Delavirdine, Dextromethorphan, Diazepam, Diclofenac, Enalapril, Erythromycin, Estradiol, Estrogen, Fentanyl, Finasteride, Flecainide, Fluoxetine, Glipizide, Glyburide, Haloperidol, Indinavir, Indomethacin, Lidocaine, Lopinavir, Loratidine, Methadone, Mexiletine, Morphine, Nelfinavir, Nifedipine, Olanzapine, Omeprazole, Opioid, Pentamidine, Phenothiazines, Phenytoin, Piroxicam, Prednisone, Progesterone, Propranolol, Quinidine, Risperidone, Ritonavir, Selective serotonin reuptake inhibitors, Saquinavir, Sildenafil, Sirolimus, Statins, Tacrine, Tacrolimus, Tamoxifen, Testosterone, Theophylline, Tramadol, Trazodone, Tricyclic, Valproate, Venlafaxine, Verapamil, or Voriconazole.

The injection system 216 can be controlled, such as by the controller 214, to inject one or more fluids into the blood circuit 204 such as to treat, grow, or support the organ 50 in the enclosure 202. Additional details of operation of the system 200 and the injection system 216 are discussed below with respect to FIG. 3.

The secondary circuit 212a can optionally include a bubble sensor 234 connected to the secondary circuit 212a and downstream of the bypass 212c and therefore downstream of all of the pumps 206, allowing bubbles to be detected upstream of the organ 50. The bubble sensor 234 can be in communication with the controller 214. A bubble trap 236 can be located in the secondary circuit 212a, such as downstream of the secondary circuit 212a and downstream of the bypass 212c and therefore downstream of all of the pumps 206, allowing bubbles created by agitation during pumping to be collected upstream of the organ 50.

The secondary circuit 212a can optionally include an inlet sensor suite 238a that can optionally include one or more of a pressure sensor, a temperature sensor, a flow meter, a glucose sensor, a lactate sensor an O2 sensor, a CO2 sensor, a pH sensor, or other sensors. Each sensor can be connected to the secondary circuit 212a upstream of the enclosure 202 (and can optionally be within the enclosure 202). Similarly, an outlet sensor suite 238b can optionally include one or more of a pressure sensor, a temperature sensor, a flow meter, a glucose sensor, a lactate sensor, an O2 sensor, a CO2 sensor, an organ health sensor, or other sensors. Each sensor can be connected to the secondary circuit 212a downstream of the enclosure 202 (and can be optionally be within the enclosure 202). Though the sensors are shown in FIG. 2 as having specific locations, the sensors can be positioned in various locations in other examples.

For example, a temperature sensor can be located upstream of the enclosure 202 or downstream of the enclosure 202. The inlet temperature sensor can be configured to produce an inlet temperature signal based on an inlet temperature of the blood entering the enclosure 202. The outlet temperature sensor can be configured to produce an outlet temperature signal based on an outlet temperature of the blood leaving the enclosure 202. The temperature sensor(s) be any type of fluid temperature sensor, either in a thermowell, coupled to a pipe of the circuit 204, or in direct contact with the process fluid, such as a thermistor, thermocouple, resistance temperature detector, or the like. The inlet sensor suite 238a or the outlet sensor suite 238b can include other sensor options, some examples of which are discussed below.

In another example, an inlet glucose sensor can be connected to the secondary circuit 212a, such as upstream of the enclosure 202. Similarly, an outlet glucose sensor can be connected to the secondary circuit 212a, such as downstream of the enclosure 202. The glucose sensors can be configured to produce a glucose sensor signal based on a glucose level of the blood. Similarly, an organ health sensor can be connected to the secondary circuit 212a, such as upstream or downstream of the enclosure 202. The organ health sensor can be configured to produce an organ health sensor signal based on a metabolite level of the blood within the circuit 204. An inlet CO2 sensor can be connected to the secondary circuit 212a upstream of the enclosure 202. An outlet CO2 sensor can be connected to the secondary circuit 212a downstream of the enclosure 202. Each of the inlet CO2 sensor and the outlet CO2 sensor can be a carbon dioxide sensor configured to produce a carbon dioxide signal based on a respective CO2 level of the blood of the blood. An inlet O2 sensor can be connected to the secondary circuit 212a upstream of the enclosure 202. An outlet O2 sensor can be connected to the secondary circuit 212a downstream of the enclosure 202. Each of the inlet O2 sensor and the O2 sensor can be an oxygen sensor configured to produce an oxygen signal based on a respective O2 level of the blood. An inlet pressure sensor can be connected to the secondary circuit 212a upstream of the enclosure 202 and can be configured to produce an inlet pressure signal based on a pressure of the blood leading into the enclosure 202. An outlet pressure sensor can be connected to the secondary circuit 212a upstream of the enclosure 202 and can be configured to produce an inlet pressure signal based on a pressure of the blood leading into the enclosure 202. Any or all of the sensor signals can be transmitted to the controller 214.

A sampling port 240a can be connected to the secondary circuit 212a (for example upstream of the pump 206a) and can be configured to receive a syringe to withdraw a sample of the blood without compromising sterility of the blood within the secondary circuit 212a. A sampling port 240b can be connected to the secondary circuit 212a (for example downstream of the pump 206a) and can be configured to receive a syringe to withdraw a sample of the blood without compromising sterility of the blood within the secondary circuit 212a. Also, the blood circuit 204 can include one or more dosing ports that can be configured to receive supplements for delivery to the blood and ultimately the organ without compromising sterility of the blood within the circuit 204.

The primary circuit 212b can optionally include a bubble sensor 242 connected to the primary circuit 212b downstream of the bypass 212c and therefore downstream of the pump 206a and upstream of the patient 54, allowing bubbles to be detected upstream of the patient 54. The bubble sensor 234 can be in communication with the controller 214. A bubble trap 244 can be located in the primary circuit 212b, such as downstream of the secondary circuit 212a and downstream of the bypass 212c and therefore downstream of the pump 206a, allowing bubbles created by agitation during pumping to be collected or trapped upstream of the patient 54.

The primary circuit 212b can also include a secondary inlet sensor suite 246a, which can include one or more of a pressure sensor, a temperature sensor, a flow meter, a glucose sensor, a lactate sensor, an O2 sensor, a CO2 sensor, a pH sensor, or other sensors. Each sensor can be connected to the primary circuit 212b upstream of the patient 54. Similarly, primary circuit 212b can a secondary outlet sensor suite 246b can optionally include one or more of a pressure sensor, a temperature sensor, a pH sensor, a flow meter, a glucose sensor, an O2 sensor, a CO2 sensor, an organ health sensor, or other sensors. Each sensor can be connected to the primary circuit 212b downstream of the patient 54. Though the sensors are shown in FIG. 2 as having specific locations, the sensors can be positioned in various locations in other examples.

The primary circuit 212b can also include a sample port 248a that can be connected to the primary circuit 212b (for example upstream of the patient 54) and can be configured to receive a syringe to withdraw a sample of the blood without compromising sterility of the blood within the primary circuit 212b. A sampling port 248b can be connected to the primary circuit 212b (for example downstream of the patient 54) and can be configured to receive a syringe to withdraw a sample of the blood without compromising sterility of the blood within the primary circuit 212b. Also, the primary circuit 212b can include a dosing port 250 that can be configured to receive supplements for delivery to the blood and ultimately the patient 54 and organ 50 without compromising sterility of the blood within the circuit 204.

The primary circuit 212b can also include a bubble sensor 252 that can be located upstream of the patient 54 and can be in communication with the controller 214. The controller 214 can be configured to shut down the system 200 if the bubble sensor 252 detects a bubble to help protect the health and safety of the patient 54.

FIG. 3 illustrates a schematic view of the system 200 for support of the patient 54 using a bio-engineered organ. The system 200 can be the same or similar to the system 200 discussed above with respect to FIG. 2; FIG. 3 shows how various components can be connected to the controller 214.

The pump 206, the gas transfer unit 208, the cooling system 210, the primary sensor suites 246, the secondary sensor suites 238, the injection system 216, and the control valves 224 can be connected to the controller. The cooling system 228 and the pumps 230 can be connected directly the controller 214 or can be connected to the controller 214 through a controller or other device of the injection system 216.

The user interface 254 can be any display and/or input device. For example, user interface 254 can be a touch screen display, computer, tablet, phone, or the like. In another example, user interface 254 can provide lights, buttons, and/or switches. The user interface 254 can be in communication with the controller 214 and configured to operate the controller 214 and devices connected thereto.

One or more of the various sensors and signals of the system 200 can be used by the controller 214 to support the organ 50 within the system 200 and therefore the patient 54 in an automated or semi-automated fashion, such as to help reduce time and labor required to support the organ 50. The controller 214 can use various algorithms (e.g., PID loops) incorporating data from one or more sensors to perform such growth or maintenance of the organ. Various examples are discussed in further detail below.

In one example, the controller 214 can be configured to receive an inlet temperature signal from the inlet sensor suite 238a or the outlet temperature signal from the outlet sensor suite 238b. The controller 214 can further be configured to operate the pumps 206, the gas transfer unit 208, or the heating and cooling system 210 based on the temperature signals. For example, the controller 214 can activate the cooling of the system 210 when the outlet temperature signal indicates that a temperature of the blood in the circuit 204 is above a threshold, for example 37, 38, or 39 degrees Celsius. Similarly, the controller 214 can activate heating of the system 210 when the outlet temperature signal indicates that a temperature of the blood in the circuit 204 is below a threshold, for example 37, 36, or 35 degrees Celsius. This control can help to ensure fluid entering the patient 54 is at an acceptable level and for the organ 50. In some examples, the controller 214 can modify operation of such components based on an ambient temperature signal from an ambient temperature sensor. The controller 214 can also be configured to produce an alert based on any of the temperature sensor signals.

In another example, the controller 214 can be configured to receive a glucose sensor signal from a glucose sensor (such as of the inlet sensor suite 238a or the outlet sensor suite 238b) and the controller 214 can be configured to operate the pump 206, the gas transfer unit 208, or the injection system 216 based on the glucose sensor signal. For example, if a glucose concentration of the blood in the circuit 204 drops below a threshold (for example 0.5 grams per liter), the controller 214 can activate a glucose injection pump 230 to inject glucose into the blood circuit 204. The controller 214 can use one or more glucose sensor signals to determine a glucose consumption rate of the organ 50 or the patient 54. The controller 214 can also be configured to adjust glucose injection based on consumption or can produce an alert based on the glucose consumption, such as if a glucose consumption rate of the organ 50 or the patient 54 drops below a threshold (for example 100 milligrams per hour).

In another example, the controller 214 can be configured to receive a pressure signal from a pressure sensor (such as of the sensor suites 238 or 240) and the controller 214 can be configured to operate the pumps 206a, 206b, 206c, or the cooling system 210 based on the pressure signal. The controller 214 can also be configured to produce an alert based on the pressure signal, such as if the pressure drops below a threshold pressure and the pump is operating, indicating that the pump is failing or has failed. The pumps 206 can be controlled to vary a flow rate of blood through the blood circuit 204, such as between 0 and 2300 milliliters per minute (ml/min). Optionally, the pumps 206a and 206b can operate between 0 and 150 ml/min and the pump 206c can operate around 1000 ml/min, such as to provide a higher flow rate in the secondary circuit 212a. The pumps 206 can also be controlled to vary an operating pressure, such as between 0 and 200 millimeters of Mercury (mm/Hg). Flow of the pumps 206 can be controlled based on monitored pressure of the secondary circuit 212a or the primary circuit 212b.

For example, the controller 214 can monitor flow rates and pressures through the secondary circuit 212a using one or more of the inlet sensor suite 238a or the outlet sensor suite 238b and can adjust flow of one or more of the pumps 206 based on the pressure signals or flow signals such as to maintain a desired pressure or flow rate through the organ 50. Similarly, the controller 214 can monitor flow rates and pressures through the primary circuit 212b using one or more of the secondary inlet sensor suite 246a and the secondary outlet sensor suite 246b and can adjust flow of one or more of the pumps 206 based on the pressure signals or flow signals such as to maintain a desired pressure or flow rate through the patient 54 or to match a flow rate or fluid pressure of the patient.

In another example, the controller 214 can be configured to receive a gas signal from a gas sensor (such as of the inlet sensor suite 238a or the outlet sensor suite 238b) and can use the gas signal (e.g., O2 or CO2) to determine gas consumption of the organ 50 based on the gas signal. The controller 214 can also operate the gas transfer unit 208 or the gas tank 222 based on the determined gas consumption of the organ 50 or can produce an alert based on the determined consumption. Similarly, the controller 214 can be configured to receive a gas signal from a gas sensor (such as of the secondary inlet sensor suite 246a or the secondary outlet sensor suite 246b) and can use the gas signal (e.g., O2 or CO2) to determine gas consumption of the patient 54 based on the gas signal. The controller 214 can also operate the gas transfer unit 208 or the gas tank 222 based on the determined gas consumption of the patient 54 or can produce an alert based on the determined consumption. The controller 214 can also operate the injection system 216 based on the gas signal. For example, the controller 214 can inject one or more solutions based on the consumption rate of the patient 54 or the organ 50.

In another example, the controller 214 can be configured to receive a pH signal from a pH sensor (such as of the inlet sensor suite 238a or the outlet sensor suite 238b) and can use the pH signal to determine a pH range of the blood in the secondary circuit 212a. The controller 214 can operate one or more of the pumps 230 of the injection system 216 to provide one or more fluids to the blood circuit 204 to maintain the pH in a desired or operable range. Similarly, the controller 214 can be configured to receive a pH signal from a pH sensor of the secondary inlet sensor suite 246a or the secondary outlet sensor suite 246b and can use the pH signal(s) to determine a pH range of the blood in the primary circuit 212b. The controller 214 can operate one or more of the pumps 230 of the injection system 216 to provide one or more fluids to the blood circuit 204 to maintain the pH in a desired or operable range. The controller 214 can also be configured to produce an alert based on the pH signal, such as if the pH levels in the blood circuit 204 falls outside an acceptable range. In such an example, the alert may indicate that the organ 50 is contaminated or compromised or the organ of the patient 54 is not performing as it should.

The controller 214 can be in communication with the bubble sensors 234, 242, and 252, and can be configured to receive a bubble sensor signal from each based on detection of a bubble in the blood circuit 204. The controller 214 can shut down one or more of the pumps when a bubble is detected in the circuit. Optionally, the controller 214 can shut down only some of the pumps when a bubble is detected. For example, the pumps 206a and 206b can be shut down when a bubble is detected at the sensor 242 or 252 but the pump 206c can continue to operate, such as to continue to support the organ 50.

In another example, the controller 214 can be configured to receive the organ health sensor signal from an organ health sensor (such as of the inlet sensor suite 238a, the outlet sensor suite 238b, the secondary inlet sensor suite 246a, or the secondary outlet sensor suite 246b) and the controller 214 can be configured to operate the pumps 206 and the gas transfer unit 208 based on the organ health sensor signal. Optionally, the controller 214 can operate the injection system 216 to operate one or more of the pumps 230 to pump a fluid from one or more of the tanks 232 to the blood circuit 204 based on the signal(s) from the organ health sensor(s). The controller 214 can also be configured to produce an alert based on the organ health sensor signal. In some examples, the organ health sensors can be a metabolite sensor or sensors.

The controller 214 can operate the pumps 230 to inject one or more nutrients to the blood circuit 204 to maintain the desired metabolite levels of the organ 50, the patient 54, and the blood circuit 204. The organ health sensor can be various types of sensors configured to monitor one or more conditions of the blood. For example, the organ health sensor can be an ammonia sensor configured to produce a signal based on an ammonia concentration or level of the blood. Also, the organ health sensor can be a glutamine sensor configured to produce a signal based on a glutamine concentration or level of the blood. Also, the organ health sensor can be a lactate sensor configured to produce a signal based on a lactate concentration or level of the blood. Also, the organ health sensor can be a bile sensor configured to produce a signal based on a bile concentration or level of the blood. Also, the organ health sensor can be a creatinine sensor configured to produce a signal based on a creatinine concentration or level of the blood. Also, the organ health sensor can be an albumin sensor configured to produce a signal based on an albumin concentration or level of the blood. Also, the organ health sensor can be a coagulation time sensor to produce a signal based on an active coagulation time (ACT) of the blood. Clotting factors, such as (Factor VII, Factor VIII, or Factor X), can also be monitored in blood, blood, or media. In some examples, the controller 214 can be configured to receive the health signal from the health sensor and can be configured to control an injection of an anticoagulant (such as heparin) if the ACT falls below a threshold (such as below 100 seconds).

In some examples, the sampling port 240a can be used to take a sample volume out of the blood circuit. The sample can be manually tested and the sample can be automatically tested for sampling and analysis during transport. Samples collected from the sampling port 240a can be used by the controller 214 to add biochemical components through the injection system 216 that are necessary to develop and maintain the function of the organ 50 or the organ of the patient 54. Further, automatic sampling of sub-systems can be performed by the controller 214. For example, blood can be automatically drawn from the system and can be delivered to a component that measures ACT, glucose, lactate, ammonia, or the like.

In some examples, samples of the blood or media can be taken through the sampling port 240a (or using a glucose sensor of the secondary sensor suite 238a). The samples can be assayed (such as by the controller 214) for glucose content of the blood over a specified period of time. This can be done in order to determine if the organ 50 or the patient 54 has the adequate glucose consumption rate. When it is determined that the glucose level of the blood in the circuit 204 is too low, the controller 214 can operate the injection system 216 to inject glucose into the circuit 204 to help maintain the organ 50 and the organ of the patient 54. In some examples, multiple samples can be used from multiple sampling ports (such as the sampling port 240a and the sample port 248a) to determine which organ may be operating sub-optimally.

In some examples, a specified dose of drugs commonly cleared in the organ can be injected over a specified period of time ((R)-Warfarin, (S)-Mephenytoin, Acetaminophen, Aprepitant, Azole, Benzodiazepines, Beta, Caffeine, Calcium, Carbamazepine, Celecoxib, Clarithromycin, Codeine, Cyclosporine, Delavirdine, Dextromethorphan, Diazepam, Diclofenac, Enalapril, Erythromycin, Estradiol, Estrogen, Fentanyl, Finasteride, Flecainide, Fluoxetine, Glipizide, Glyburide, Haloperidol, Indinavir, Indomethacin, Lidocaine, Lopinavir, Loratidine, Methadone, Mexiletine, Morphine, Nelfinavir, Nifedipine, Olanzapine, Omeprazole, Opioid, Pentamidine, Phenothiazines, Phenytoin, Piroxicam, Prednisone, Progesterone, Propranolol, Quinidine, Risperidone, Ritonavir, Selective serotonin reuptake inhibitors, Saquinavir, Sildenafil, Sirolimus, Statins, Tacrine, Tacrolimus, Tamoxifen, Testosterone, Theophylline, Tramadol, Trazodone, Tricyclic, Valproate, Venlafaxine, Verapamil, or Voriconazole.). Samples of the blood or media can be taken from the sampling port 240a periodically and can be bioanalyzed to determine if the liver BEO (the organ 50) is clearing the drugs to at an adequate rate. In some examples, samples of the blood or media can be taken from the sampling port 240a and assayed for albumin content over a specified period of time in order to determine if the liver BEO has adequate albumin production. In some examples, samples of the blood or media can be taken from the sampling port 240a and assayed for bile content over a specified period of time in order to determine if the liver BEO has adequate bile production. In some examples, samples of the blood or media can be taken from the sampling port 240a and can be assayed for creatinine, BSA, and/or urea content over a specified period of time in order to determine if the kidney BEO (the organ 50) has the glomerular filtration rate indicative of normal organ operation or function.

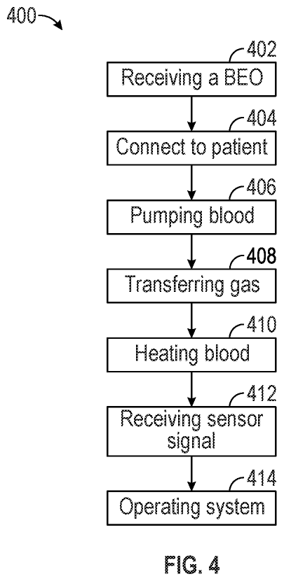

FIG. 4 illustrates a schematic view of a method 400, in accordance with at least one example of the present disclosure. The method 400 can be a method of testing and supporting a BEO and a patient. The steps or operations of the method 400 are illustrated in a particular order for convenience and clarity; many of the discussed operations can be performed in a different sequence or in parallel without materially impacting other operations. The method 400 as discussed includes operations performed by multiple different actors, devices, and/or systems. It is understood that subsets of the operations discussed in the method 400 can be attributable to a single actor, device, or system could be considered a separate standalone process or method.

Method 400 can begin at step 402, where an organ can be received in an enclosure, where the enclosure includes a blood inlet and a blood outlet connected to a secondary loop of the system. For example, the organ 50 can be received into the enclosure 202 connected to the secondary circuit 212a. At step 404, a primary loop of the system can be connected to a patient, where the primary loop is connected to the secondary loop. For example, the primary circuit 212b of the system 200 can be connected the a patient 54. At step 406, blood can be pumped through the primary loop using a primary pump to transmit blood through the patient. For example, blood can be pumped through the primary circuit 212b using the pumps 206a and 206b to transmit blood through the patient 54. Also, at step 406, blood can be pumped through the secondary loop using a secondary pump, where the secondary pump can be connected to the blood inlet and the blood outlet to transmit blood through the organ and the primary loop. For example, blood can be pumped through the secondary circuit 212a using the pump 206c to transmit blood through the organ and the secondary circuit 212a.

At step 408, gas can be transferred to or from the blood using a gas transfer unit connected to the blood circuit. For example, gas can be transferred to or from the blood of the blood circuit 204 using the gas transfer unit 208. At step 410, blood of the circuit 204 can be heated. At step 412, a sensor signal can be received, where the sensor signal is indicative of a condition of the blood. For example, the controller 214 can receive a signal from one or more of the sensors of the inlet sensor suite 238a, the outlet sensor suite 238b, the secondary inlet sensor suite 246a, or the secondary outlet sensor suite 246b. At step 414 the pump and the injection system can be operated based on one or more of sensor signals.

In another example, nutrients can be injected into the system using an injection system connected to the secondary circuit. For example, nutrients can be injected into the system 200 using the injection system 216 connected to the secondary circuit 212a.

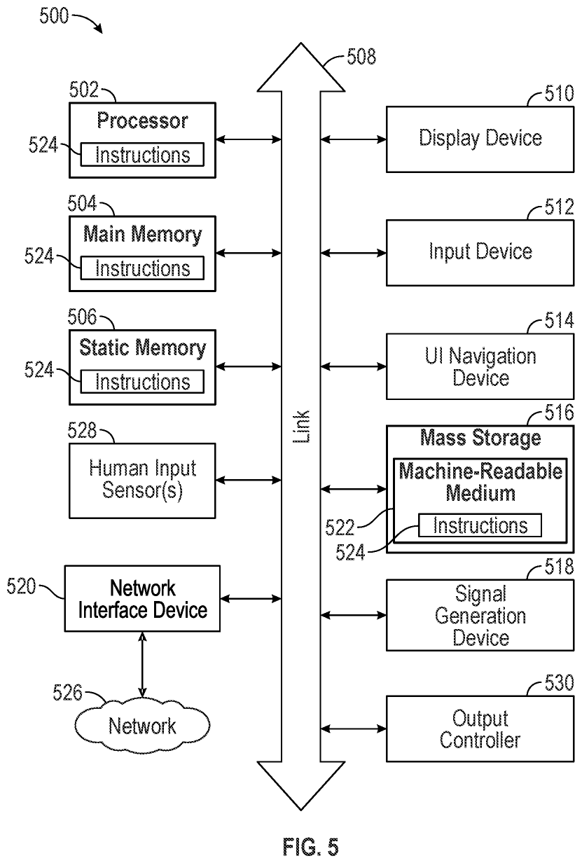

FIG. 5 illustrates a block diagram of an example machine 500 upon which any one or more of the techniques (e.g., methodologies) discussed herein can perform. Examples, as described herein, can include, or can operate by, logic or a number of components, or mechanisms in the machine 500 (which can be the system 200). Circuitry (e.g., processing circuitry) is a collection of circuits implemented in tangible entities of the machine 500 that include hardware (e.g., simple circuits, gates, logic, etc.). Circuitry membership can be flexible over time. Circuitries include members that may, alone or in combination, perform specified operations when operating. In an example, hardware of the circuitry can be immutably designed to carry out a specific operation (e.g., hardwired). In an example, the hardware of the circuitry can include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components can be used in more than one member of more than one circuitry. For example, under operation, execution units can be used in a first circuit of a first circuitry at one point in time and reused by a second circuit in the first circuitry, or by a third circuit in a second circuitry at a different time. Additional examples of these components with respect to the machine 500 follow.

In alternative embodiments, the machine 500 can operate as a standalone device or can be connected (e.g., networked) to other machines. In a networked deployment, the machine 500 can operate in the capacity of a server machine, a client machine, or both in server-client network environments. In an example, the machine 500 can act as a peer machine in peer-to-peer (P2P) (or other distributed) network environment. The machine 500 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 500 can include a hardware processor 502 (e.g., a central processing unit (CPU), a graphics processing unit (GPU), a hardware processor core, or any combination thereof), a main memory 504, a static memory (e.g., memory or storage for firmware, microcode, a basic-input-output (BIOS), unified extensible firmware interface (UEFI), etc.) 506, and mass storage 508 (e.g., hard drive, tape drive, flash storage, or other block devices) some or all of which can communicate with each other via an interlink (e.g., bus) 530. The machine 500 can further include a display unit 510, an alphanumeric input device 512 (e.g., a keyboard), and a user interface (UI) navigation device 514 (e.g., a mouse). In an example, the display unit 510, input device 512 and UI navigation device 514 can be a touch screen display. The machine 500 can additionally include a storage device (e.g., drive unit) 508, a signal generation device 518 (e.g., a speaker), a network interface device 520, and one or more sensors 516, such as a global positioning system (GPS) sensor, compass, accelerometer, or other sensor. The machine 500 can include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 502, the main memory 504, the static memory 506, or the mass storage 508 can be, or include, a machine readable medium 522 on which is stored one or more sets of data structures or instructions 524 (e.g., software) embodying or utilized by any one or more of the techniques or functions described herein. The instructions 524 can also reside, completely or at least partially, within any of registers of the processor 502, the main memory 504, the static memory 506, or the mass storage 508 during execution thereof by the machine 500. In an example, one or any combination of the hardware processor 502, the main memory 504, the static memory 506, or the mass storage 508 can constitute the machine readable media 522. While the machine readable medium 522 is illustrated as a single medium, the term “machine readable medium” can include a single medium or multiple media (e.g., a centralized or distributed database, and/or associated caches and servers) configured to store the one or more instructions 524.

The term “machine readable medium” can include any medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Non-limiting machine readable medium examples can include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media can include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 can be further transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 520 can include one or more physical jacks (e.g., Ethernet, coaxial, or phone jacks) or one or more antennas to connect to the communications network 526. In an example, the network interface device 520 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software. A transmission medium is a machine readable medium.

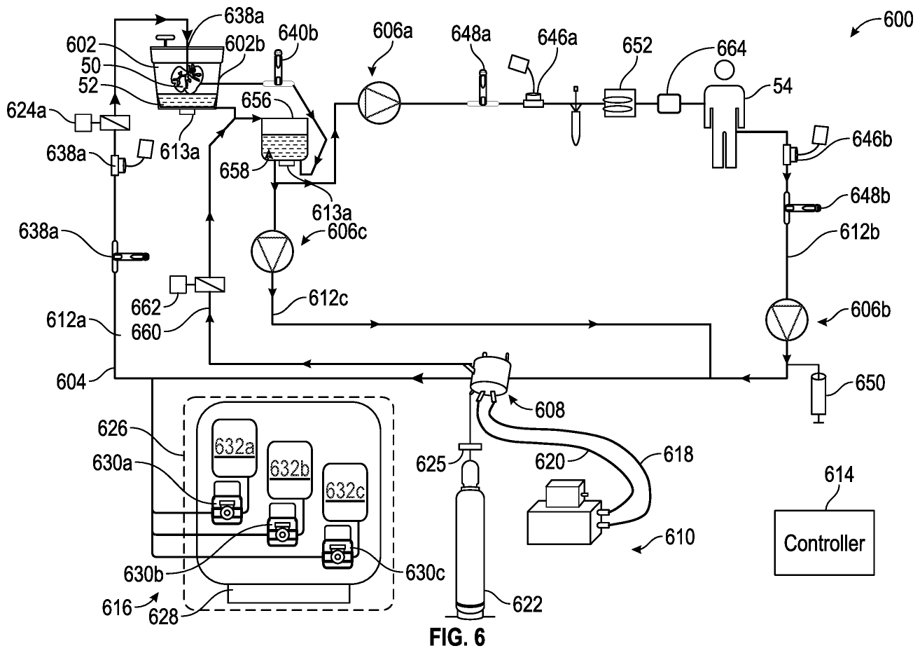

FIG. 6 illustrates a schematic view of a system 600 for growth and support of a bio-engineered organ. The system 600 can be similar to the system 200 discussed above; the system 600 can include additional features or components, such as a storage container and a purge line. Any of the systems discussed above or below can be modified to include the features of the system 600. In the system 600, components with reference numerals similar to those of the system 200 can reference similar components that can be connected and can operate as described above.

More specifically, the system 600 can include a storage container 656. The storage container 656 can be connected to a blood circuit 604 at a downstream side of an enclosure 602 and can be independently or separately connected to a downstream side of the organ 50, such as to one or more vessels of the organ 50. In this way, blood flowing from a drain of the enclosure 602 and through the organ 50 can be reintroduced into the circuit via the storage container 656.

The drain of the enclosure 602 can be gravity fed and can therefore connect to a bottom of the enclosure 602 and a top portion of the storage container 656. This setup can allow the storage container 656 to help maintain a desired volume of the blood within the enclosure 602. Because the storage container 656 can be relatively smaller than the enclosure 602 (or can be shaped differently), the storage container 656 can create a relatively smaller surface area for contact between air and blood within the storage container 656 than in the enclosure 602. Therefore, the storage container 656 to maintain a low volume of the blood within the enclosure 602, which can help to limit exposure of the blood to air. The blood outlet 602b from the organ 50 can be connected to a bottom or other portion of the storage container 656 and can be driven to flow by one or more of a primary pump 606a and a secondary pump 606c.

The system 600 can also include a weight sensor 613a connected to the enclosure 602, similar to the sensor 213 discussed above. The system 600 can further include a weight sensor 613b connected to the storage container 656. The weight sensor 613b can be in communication with the controller 614 such as to transmit a weight signal thereto. The controller 614 can determine a weight or volume of fluid within the storage container 656 and can optionally control various components of the system based on the weight or volume, such as the pumps 606. This can allow the controller 614 to target a specific volume or weight of the storage container 656, helping to limit overfilling or depletion of the fluid (e.g., blood) within storage container 656.

A discharge of the storage container 656 can be connected to an upstream side or an inlet of both the primary pump 606a and the secondary pump 606c such that the storage container 656 can provide a buffer volume of blood or fluid to supply the primary pump 606a or the secondary pump 606c. The primary pump 606a and a second primary pump 606b can each be in communication with a controller 614 such that the controller 614 can operate the pumps 606a and 606b (which can be in series with each other but located on opposite sides of the patient 54) such that blood volume to and from the patient is net zero during normal operation of the system 600.

The system 600 can include a filter 658 located at least partially within the storage container 656. The filter 658 can be configured to filter impurities, blood clots, or contaminates from the blood as it passes through the storage container 656. For example, the filter 658 can be located between the inlets from the enclosure 602 and the organ 50 and the outlet connected to the pumps 606a and 606c.

FIG. 6 also shows that the system 600 can include a purge line 660 connected to a gas transfer unit 608 unit and connected to the storage container 656. The purge line 660 can be configured to transmit blood from the gas transfer unit 608 during a flushing or purge sequence, such as during a portion of a startup sequence. The purge line 660 can help carry trapped gasses, such as oxygen, within a blood volume portion of the gas transfer unit 608 from the gas transfer unit 608 into the storage container 656 to allow the gasses to be vented from the system 600 or collected in the storage container 656. This process can help ensure that the blood volume of the gas transfer unit 608 is primed with blood, helping to limit opportunity for gas bubbles to make their way into the circuit. A purge valve 662 can be connected to the purge line 660 and can be in communication with the controller 614. The controller 614 can operate the purge valve 662 to control flow through the purge line 660 during the purge sequence and can help to ensure that blood does not flow through the purge line 660 during normal operation, such as by closing the purge valve 662 during normal operation where the organ 50 or patient 54 is supported.

The system 600 can also include one or more tube clamps 664 connected to a primary circuit 612b, such as for connecting and disconnecting the patient 54 from the primary circuit 612b.

The system 600 can be configured to support several types of organs within the enclosure 602 and thereby of the patient 54 when the system 600 is connected to the patient. For example, the system 600 can be configured to support a liver of the patient 54, such as where the organ 50 is a liver and the primary circuit 612b is connected to a liver of the patient 54. In such an example, the system 600 can be configured to support and monitor the liver of the patient such as by the organ 50 operating to perform functions of a liver in place of or in support of the liver of the patient 54. For example, the liver 50 can process ammonia, lipids, or glucose.

For example, when the organ 50 is helping to support or monitor ammonia clearance or removal of the liver of the patient 54, ammonia levels can be monitored in the secondary circuit 612a and the primary circuit 612b, such as via an inlet sensor suite 638a, which can be in communication with the controller 614. The sensor(s) can transmit an ammonia sensor signal to the controller 614 and the controller 614 can use the signal(s) to determine an amount of ammonia within the blood of the circuit. The controller 614 can operate the gas transfer unit 608, an injection system 616, and one or more of the pumps based on the detected or determined amount of ammonia within the system 600.

Similarly, the inlet sensor suite 638a can include one or more sensors for allowing the controller 614 to determine Prothrombin time (PT) or international normalized ration (INR) and the controller 614 can operate the gas transfer unit 608, an injection system 616, and one or more of the pumps based on the detected or determined PT or INR within the system 600 or of the patient 54.

The system 600 can also include sensors for determining or detecting levels of albumin, urea, cholesterol, or proteins to determine a general state of the system 600 or the patient 54 or for detecting or determining specific conditions that may indicate health or performance of the liver. The controller 614 can operate the gas transfer unit 608, an injection system 616, and one or more of the pumps based on the detected or determined conditions within the system 600.

During conditioning or support of a liver within the patient 54, the controller 614 can operate the second primary pump 606b at a rate of between 0-300 milliliters per minute (ml/min) on pump 1 and can control the secondary pump 606c to a rate of between 200 to 2000 ml/min.

The system 600 can also be configured to support other organs of a patient, such as a kidney, pancreas, spleen, or heart. For example, when the organ 50 is a biologically engineered kidney and the system 600 is connected to a kidney of a patient, the system 600 can detect or determine kidney indicators, such as creatinine, blood urea nitrogen (BUN), albumin, or other proteins, and the controller 614 can operate the gas transfer unit 608, an injection system 616, and one or more of the pumps based on the detected or determined kidney conditions within the system 600.

### NOTES AND EXAMPLES

The following, non-limiting examples, detail certain aspects of the present subject matter to solve the challenges and provide the benefits discussed herein, among others.

Example 1 is a system for supporting a patient organ, the system comprising: a primary circuit including an inlet configured to connect to the patient and an outlet configured to connect to the patient, the primary circuit comprising: a primary pump configured to circulate blood through the primary circuit and into the patient; a secondary circuit connected to the primary blood circuit, the secondary circuit comprising: an enclosure configured to support a organ therein in a blood flow, the enclosure including a blood inlet and a blood outlet to receive the blood flow through the enclosure and through the organ; a secondary pump configured to circulate blood through the secondary circuit; a gas transfer unit configured to transfer gas to and from the blood; a secondary sensor connected upstream of the enclosure and configured to produce a secondary sensor signal based on a condition of the blood; a controller configured to: operate the gas transfer unit based on the secondary sensor signal.

In Example 2, the subject matter of Example 1 optionally includes a bypass circuit connected to the primary circuit and the secondary circuit, the bypass circuit; and a second primary pump located downstream of the patient, the second primary pump configured to circulate blood through the primary circuit and from the patient.

In Example 3, the subject matter of any one or more of Examples 1-2 optionally include the secondary circuit comprising: a port upstream of the enclosure for sampling of the blood.

In Example 4, the subject matter of any one or more of Examples 1-3 optionally include a gas mixture unit connected to the gas transfer unit and configured to deliver gas to the gas transfer unit.

In Example 5, the subject matter of Example 4 optionally includes wherein the secondary sensor includes a pressure transducer configured to transmit a pressure signal to the controller based on a pressure of the blood and a temperature sensor configured to transmit a temperature signal to the controller based on a temperature of the blood, the controller to operate the gas transfer unit, the primary pump, and the secondary pump based on the pressure signal and the temperature signal.

In Example 6, the subject matter of Example 5 optionally includes the secondary circuit comprising: an inlet dissolved oxygen sensor upstream of the enclosure and configured to transmit an inlet oxygen signal to the controller based on an inlet dissolved oxygen level of the blood; and an outlet dissolved oxygen sensor downstream of the enclosure and configured to transmit an outlet oxygen signal to the controller based on an outlet dissolved oxygen level of the blood.

In Example 7, the subject matter of Example 6 optionally includes wherein the controller is configured to determine an oxygen use rate of the organ based on the inlet oxygen signal and the outlet oxygen signal, and wherein the controller is configured to operate the gas transfer unit, the primary pump, and the secondary pump based on the oxygen use rate of the organ.

In Example 8, the subject matter of Example 7 optionally includes wherein the controller is configured to determine an oxygen use rate of the patient organ based on the inlet oxygen signal and the outlet oxygen signal, and wherein the controller is configured to operate the gas transfer unit, the primary pump, and the secondary pump based on the oxygen use rate of the patient organ.

In Example 9, the subject matter of any one or more of Examples 2-8 optionally include the secondary circuit comprising: a bubble trap and a bubble sensor upstream of the gas transfer unit.

In Example 10, the subject matter of any one or more of Examples 1-9 optionally include wherein the gas transfer unit is an oxygenator.

In Example 11, the subject matter of Example 10 optionally includes wherein the gas transfer unit includes an air separator.

In Example 12, the subject matter of any one or more of Examples 2-11 optionally include a heating system connected to the gas transfer unit to exchange heat with the blood through the gas transfer unit.

In Example 13, the subject matter of any one or more of Examples 1-12 optionally include an injection system connected to the secondary circuit upstream of the enclosure and in communication with the controller, the controller configured to operate the injection system based on the sensor signal to deliver supplements to the blood.

In Example 14, the subject matter of Example 13 optionally includes wherein the injection system includes a plurality of injection pumps each configured to deliver a supplement to the blood, the controller configured to operate each of the injection pumps based on the secondary sensor signal to deliver supplements to the blood.

In Example 15, the subject matter of Example 14 optionally includes S), Sodium Bicarbonate, Insulin, Epinephrine, Albumin, linoleic acid, dexamethasone, and glucagon.

In Example 16, the subject matter of Example undefined optionally includes, wherein the injection system includes an enclosure supporting the plurality of injection pumps and the supplements.

In Example 17, the subject matter of Example 16 optionally includes wherein the injection system includes a cooling system configured to cool an environment of the injection system, the cooling system in communication with the controller, the controller configured to operate the cooling system to maintain a desired temperature of the environment of the injection system.

In Example 18, the subject matter of Example undefined optionally includes, further comprising: a storage container connected to a discharge of the enclosure and connectable to a discharge of a vessel of the organ, the storage container located upstream of the secondary pump and the primary pump.

In Example 19, the subject matter of Example 18 optionally includes a filter located at least partially within the storage container and configured to filter perfusate passing through the storage container.

In Example 20, the subject matter of any one or more of Examples 18-19 optionally include a purge line connected to the gas transfer unit and connected to the storage container.

Example 21 is a method for growing or supporting a organ using a system, the method comprising: receiving an organ in an enclosure, the enclosure including a blood inlet and a blood outlet connected to a secondary loop of the system; connecting a primary loop of the system to a patient, the primary loop connected to the secondary loop; pumping blood through the primary loop using a primary pump to transmit blood through the patient; pumping blood through the secondary loop using a secondary pump, the secondary pump connected to the blood inlet and the blood outlet to transmit blood through the organ and the secondary loop; transferring gas to or from the blood using a gas transfer unit connected to a blood circuit including the primary loop and the secondary loop; receiving a sensor signal indicative of a condition of the blood; and operating the pump based on the sensor signal.

In Example 22, the subject matter of Example 21 optionally includes injecting nutrients into the system using an injection system connected to the secondary circuit.

In Example 23, the subject matter of Example 22 optionally includes wherein the nutrients are injected based on the sensor signal.

In Example 23, the subject matter of Example 21 optionally includes operating a gas mixture unit to deliver gas to the gas transfer unit.

In Example 24, the subject matter of Example 23 optionally includes wherein the gas mixture unit delivers one or more of Carbon Dioxide, Dioxygen, Nitrogen, or Argon to the gas transfer unit based on the sensor signal.

In Example 25, the subject matter of any one or more of Examples 18-20 optionally include injecting nutrients into the system using an injection system connected to the secondary circuit.

Example 26 is at least one machine-readable medium including instructions that, when executed by processing circuitry, cause the processing circuitry to perform operations to implement of any of Examples 1-25.

Example 27 is an apparatus comprising means to implement of any of Examples 1-25.

Example 28 is a system to implement of any of Examples 1-25.

Example 29 is a method to implement of any of Examples 1-25.

In Example 30, the apparatuses or method of any one or any combination of Examples 1-29 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system for supporting a patient organ, the system comprising:
a primary circuit including an inlet configured to connect to the patient and an outlet configured to connect to the patient, the primary circuit comprising:
a primary pump configured to circulate blood through the primary circuit and into the patient;
a secondary circuit connected to the primary blood circuit, the secondary circuit comprising:
an enclosure configured to support an organ therein in a blood flow, the enclosure including a blood inlet and a blood outlet to receive the blood flow through the enclosure and through the organ;
a secondary pump configured to circulate blood through the secondary circuit;
a gas transfer unit configured to transfer gas to and from the blood;
a secondary sensor connected upstream of the enclosure and configured to produce a secondary sensor signal based on a condition of the blood; and
a controller configured to:
operate the gas transfer unit based on the secondary sensor signal.

2. The system of claim 1, further comprising:
a bypass circuit connected to the primary circuit and the secondary circuit, the bypass circuit; and
a second primary pump located downstream of the patient, the second primary pump configured to circulate blood through the primary circuit and from the patient.

3. The system of claim 1, the secondary circuit comprising:
a port upstream of the enclosure for sampling of the blood.

4. The system of claim 1, further comprising:
a gas mixture unit connected to the gas transfer unit and configured to deliver gas to the gas transfer unit.

5. The system of claim 4 wherein the secondary sensor includes a pressure transducer configured to transmit a pressure signal to the controller based on a pressure of the blood and a temperature sensor configured to transmit a temperature signal to the controller based on a temperature of the blood, the controller to operate the gas transfer unit, the primary pump, and the secondary pump based on the pressure signal and the temperature signal.

6. The system of claim 5, the secondary circuit comprising:
an inlet dissolved oxygen sensor upstream of the enclosure and configured to transmit an inlet oxygen signal to the controller based on an inlet dissolved oxygen level of the blood; and
an outlet dissolved oxygen sensor downstream of the enclosure and configured to transmit an outlet oxygen signal to the controller based on an outlet dissolved oxygen level of the blood.

7. The system of claim 6, wherein the controller is configured to determine an oxygen use rate of the organ based on the inlet oxygen signal and the outlet oxygen signal, and wherein the controller is configured to operate the gas transfer unit, the primary pump, and the secondary pump based on the oxygen use rate of the organ.

8. The system of claim 7, wherein the controller is configured to determine an oxygen use rate of the patient organ based on the inlet oxygen signal and the outlet oxygen signal, and wherein the controller is configured to operate the gas transfer unit, the primary pump, and the secondary pump based on the oxygen use rate of the patient organ.

9. The system of claim 2, the secondary circuit comprising:
a bubble trap and a bubble sensor upstream of the gas transfer unit.

10. The system of claim 1, wherein the gas transfer unit is an oxygenator.

11. The system of claim 10, wherein the gas transfer unit includes an air separator.

12. The system of claim 2, further comprising:
a heating system connected to the gas transfer unit to exchange heat with the blood through the gas transfer unit.

13. The system of claim 1, further comprising:
an injection system connected to the secondary circuit upstream of the enclosure and in communication with the controller, the controller configured to operate the injection system based on the sensor signal to deliver supplements to the blood.

14. The system of claim 13, wherein the injection system includes a plurality of injection pumps each configured to deliver a supplement to the blood, the controller configured to operate each of the injection pumps based on the secondary sensor signal to deliver supplements to the blood.

15. The system of claim 14, wherein the plurality of injection pumps are each configured to deliver to the blood and the organ, one or more of cell culture media, cells, glutamine, glucose, buffer, sodium chloride, essential amino acids, non-essential amino acids, drugs, ammonia (ammonium chloride), HEPES (C₈H₁₈N₂O₄S), Sodium Bicarbonate, Insulin, Epinephrine, Albumin, linoleic acid, dexamethasone, and glucagon.

16. The system of claim 15, wherein the injection system includes an enclosure supporting the plurality of injection pumps and the supplements.

17. The system of claim 16, wherein the injection system includes a cooling system configured to cool an environment of the injection system, the cooling system in communication with the controller, the controller configured to operate the cooling system to maintain a desired temperature of the environment of the injection system.

18. The system of claim 1, further comprising:
a storage container connected to a discharge of the enclosure and connectable to a discharge of a vessel of the organ, the storage container located upstream of the secondary pump and the primary pump.

19. The system of claim 18, further comprising:
a filter located at least partially within the storage container and configured to filter perfusate passing through the storage container.

20. The system of claim 18, further comprising:
a purge line connected to the gas transfer unit and connected to the storage container.

21-23. (canceled)
